(19) United States
(12) Patent Application Publication
(10) Pub. No.: US 2023/0310534 A1
(43) Pub. Date: Oct. 5, 2023

(54) QUENCHERS THAT SLOW, DECREASE, OR INHIBIT THE FORMATION OF CHEMIEXCITATION-INDUCED CYCLOBUTANE PYRIMIDINE DIMERS

(71) Applicants: L'OREAL, Paris (FR); Yale University, New Haven, CT (US)

(72) Inventors: Kelly Marie GEORGE, Denville, NJ (US); Zhi PAN, Ridgewood, NJ (US); Laurent MARROT, Aulnay-sous-Bois (FR); Miao WANG, Westfield, NJ (US); Douglas Edgar BRASH, Woodbridge, CT (US); Sanjay PREMI, Madison, WI (US); Leticia C.P. GONCALVES, Sao Paulo - SP (BR)

(73) Assignees: L'OREAL, Paris (FR); Yale University, New Haven, CT (US)

(21) Appl. No.: 18/118,316

(22) Filed: Mar. 7, 2023

Related U.S. Application Data
(63) Continuation of application No. 15/840,580, filed on Dec. 13, 2017.

(51) Int. Cl. A61K 36/704; A61K 31/01; A61K 36/04; A61K 47/12; A61K 47/24; A61K 36/539; A61K 36/287; A61K 31/192; A61K 31/12; A61K 31/353; A61K 36/48; A61K 31/19; A61K 31/44; A61K 36/355; A61K 31/015; A61K 31/366; A61K 8/9717; A61K 8/44; A61K 8/49; A61K 8/9789; A61K 8/36; A61Q 19/00; A61K 8/365; A61K 8/31; A61K 8/40; A61K 8/35

(52) U.S. Cl. CPC A61K 36/704; A61K 31/01; A61K 36/04; A61K 47/12; A61K 47/24; A61K 36/539; A61K 36/287; A61K 31/192; A61K 31/12; A61K 31/353; A61K 36/48; A61K 31/19; A61K 31/44; A61K 36/355; A61K 31/015; A61K 31/366; A61K 8/9717; A61K 8/44; A61K 8/498; A61K 8/9789; A61K 8/4926; A61K 8/36; A61Q 19/004; A61K 8/365; A61K 8/31; A61K 8/40; A61K 8/35

(57) ABSTRACT

A method for decreasing formation of one or more chemiexcitation-induced dark cyclobutane pyrimidine dimers (dCPDs) in a cell or DNA molecule in a subject, who has been exposed to UV radiation, is provided. The method comprises administering to the subject an effective amount of a dCPD quencher within 8 hours after the exposure. The dCPD quencher is selected from the group consisting of ellagic acid, Porphyra Umbilicalis Extract (including Porphyra Umbilicalis Extract (and) Sodium Lactate (and) Lecithin), mycosporine-like amino acids (MAAs) and synthetic analogues thereof, Scutellaria Baicalensis Root Extract, Chrysanthemum Morifolium Leaf Extract, ferulic acid, fused-ring cyanoacrylates, curcumin, epigallocatechin gallate, Scutellaria Baicalensis Root Extract (and) Acacia Catechu Wood Extract, sorbic acid, Polygonum Cuspidatum Root Extract, Acacia Catechu Extract, dipicolinic acid, squalene, Lonicera Japonica (Honeysuckle) Flower Extract, beta-carotene and combinations thereof. The dCPD quencher may be administered to the subject in the absence of UV radiation.

### CROSS-REFERENCE TO RELATED APPLICATION

This application is a continuation of U.S. application Ser. No. 15/840,580 filed Dec. 13, 2017, the contents of which are incorporated herein by reference in its entireties for all purposes.

### FIELD OF THE INVENTION

The invention relates to chemical compounds that act as quenchers to prevent the formation of chemiexcitation-induced cyclobutane pyrimidine dimers, often referred to as “dark cyclobutane pyrimidine dimers” (dCPDs).

### BACKGROUND OF THE INVENTION

In melanocytes, the sunlight-induced DNA damage that causes melanoma mutations—damage in the form of cyclobutane pyrimidine dimers (CPDs)—continues to be produced for hours after exposure of ultraviolet (UV) radiation ends. The damage originates by a pathway resembling bioluminescence, but without ending in photons: UV radiation activates two enzymes that generate reactive oxygen and nitrogen species, superoxide and nitric oxide, respectively; these molecules then combine as peroxynitrite to excite an electron in a melanin degradation product, for example, a melanin fragment, to a triplet state that has the high energy of a UV photon. This process is termed “chemiexcitation.” The high energy then transfers to DNA molecules without using photons, inducing formation of CPDs in the dark, which are also referred to as dark CPDs (“dCPDs”). It has been reported that CPD are generated in melanocytes for more than 3 hours after exposure to UVA or UVB radiation. See Premi et al., Science 347(6224):842-7 (2015). It has been reported that half or more of the CPD in a melanocyte arise after UV exposure ends.

It has been proposed that the triplet state energy on a melanin degradation product may result from a triplet-state reaction intermediate formed by unstable dioxetane adducts, which undergo spontaneous thermolysis to yield two carbonyls, one of which acquires most of the energy and ends up in a high-energy triplet state. As an alternative to transferring to DNA bases to form CPDs, the triplet energy may discharge in a radiation-independent manner to sodium 9,10-dibromoanthracene-2-sulfonate (DBAS) to be emitted as fluorescence or to sorbate to be dissipated as isomerization and heat, or the triplet energy may discharge directly as ultraweak visible light.

Although screening methods have been developed to detect DNA damage and/or repair or potential active compounds/therapies/treatments to enable skin/DNA repair, there remains a need for dCPD quenchers that slow, decrease or inhibit formation of dCPDs.

### SUMMARY OF THE INVENTION

The present invention relates to a method for decreasing formation of chemiexcitation-induced dark cyclobutane pyrimidine dimers (dCPDs) in DNA molecules.

A method for decreasing formation of chemiexcitation-induced dark cyclobutane pyrimidine dimers (dCPDs) in a cell or DNA molecule in a subject who has been exposed to UV radiation. The method comprises administering to the subject an effective amount of a dCPD quencher within 8 hours after the exposure. The dCPD quencher is selected from the group consisting of ellagic acid, Porphyra Umbilicalis Extract (including Porphyra Umbilicalis Extract (and) Sodium Lactate (and) Lecithin), mycosporine-like amino acids (MAAs) and synthetic analogs thereof, Scutellaria Baicalensis Root Extract, Chrysanthemum Morifolium Leaf Extract, ferulic acid, fused-ring cyanoacrylates, curcumin, epigallocatechin gallate, Scutellaria Baicalensis Root Extract (and) Acacia Catechu Wood Extract, sorbic acid, Polygonum Cuspidatum Root Extract, Acacia Catechu Extract, dipicolinic acid, squalene, Lonicera Japonica (Honeysuckle) Flower Extract, beta-carotene and combinations thereof.

The dCPD quencher may be administered to the subject within about 1 or 3 hours after the exposure.

The dCPD quencher may be administered to the subject in the absence of UV radiation.

The method may further comprise decreasing the formation of the one or more dCPDs by at least about 40% or 80%.

The method may further comprise decreasing the formation of the one or more dCPDs within about 1 hour after the dCPD quencher is administered to the subject.

The dCPD quencher may be ellagic acid, Porphyra Umbilicalis Extract (including Porphyra Umbilicalis Extract (and) Sodium Lactate (and) Lecithin), mycosporine-like amino acids (MAAs) or synthetic analogs thereof, Scutellaria Baicalensis Root Extract, Chrysanthemum Morifolium Leaf Extract, ferulic acid, fused-ring cyanoacrylates, curcumin, epigallocatechin gallate, Scutellaria Baicalensis Root Extract (and) Acacia Catechu Wood Extract, sorbic acid, Polygonum Cuspidatum Root Extract, Acacia Catechu Extract, dipicolinic acid, squalene, Lonicera Japonica (Honeysuckle) Flower Extract, or beta-carotene.

The dCPD quencher may decrease the formation of the one or more dCPDs in the presence of peroxynitrite and melanin.

### BRIEF DESCRIPTION OF THE DRAWINGS

FIG. 1 shows quenching of light emission at 150 and 500 seconds in a peroxynitrite/melanin chemiluminescence assay to detect triplet quenchers and radical/ONOO scavengers.

FIG. 2 shows dark CPD quenching by “hits” identified in the peroxynitrite/melatonin chemiluminescence assay.

### DETAILED DESCRIPTION OF THE INVENTION

The present invention is based on the surprising discovery that only some of the highest performing peroxynitrite quenchers are capable of decreasing or slowing the formation of dark cyclobutane pyrimidine dimers (dCPDs). The inventors have developed a novel screening method for identifying dCPD quenchers or inhibitors among various test agents, including antioxidants (AOXs) such as peroxynitrite AOXs, based on their ability to decrease a chemiluminescence signal, to which a triplet energy acceptor probe such as sodium 9,10-dibromoanthracene-2-sulfonate (DBAS) diverts the triplet energy generated by oxidation of melanin by peroxynitrite. The inventors have also developed a novel validation method for identifying dCPD quenchers or inhibitors based on their ability to decrease the production of dCPDs in a DNA test target molecule. The inventors have further developed a method for decreasing the formation of dCPDs in DNA molecules with the newly identified dCPD quenchers or inhibitors. The newly identified dCPD quenchers may block the dCPD formation by quenching the high energy before the energy transfers to DNA molecules or by scavenging peroxynitrite before the triplet state is created on melanin, a related model compound such as melatonin, or a degradation product thereof.

A screening method for identifying a dark cyclobutane pyrimidine dimer (dCPD) quencher is provided. The screening method comprises incubating melanin or a related model compound such as melatonin with peroxynitrite and a triplet energy acceptor probe in a solvent in the absence of ultraviolet (UV) radiation, whereby a chemiluminescence signal is generated. The screening method further comprises adding a test agent to the solvent to decrease the chemiluminescence signal in the absence of UV radiation. The test agent does not make dCPDs. Decreasing of the chemiluminescence signal indicates that the test agent is a dCPD quencher. This screening method may be carried out in a cell-free system. The solvent may be dimethyl sulfoxide (DMSO).

The term “melanin” used herein refers to a polymer formed by melanin monomers, or a derivative thereof. A melanin derivative may be a degradation product, for example, a melanin fragment. The melanin may be eumelanin or pheomelanin. A melanin fragment may be a eumelanin or pheomelanin fragment.

The term “triplet energy acceptor probe” used herein refers to a compound, a biological molecule or a combination thereof that accepts triplet energy, for example, energy created upon oxidation of melanin by peroxynitrite, and diverts the triplet energy to generate a chemiluminescence signal. The chemiluminescence signal indicates the presence of an electronically excited triplet state, and may be quantified by single-photon counting (cpm, counts per minute). The triplet energy acceptor probe may be sodium 9,10-dibromoanthracene-2-sulfonate (DBAS).

The term “agent” used herein refers to a chemical compound, a biological molecule, or a combination thereof. The agent may be an extract from a biological organism or a part thereof. The biological organism may be a plant. A part of a plant may include leaves, a fruit, seeds, skin, or juice-all parts of the plant. The agent may be selected from the group consisting of polyphenols such as ellagic acid, epigallocatechin gallate (EGCG, also known as epigallocatechin-3-gallate) and Scutellaria Baicalensis Root Extract (and) Acacia Catechu Wood Extract; flavones such as Scutellaria Baicalensis Root Extract (and) Acacia Catechu Wood Extract, Chrysanthemum Morifolium Leaf Extract and Scutellaria Baicalensis Root Extract; cinnamate-related compounds such as curcumin, ferulic acid, Polygonum Cuspidatum Root Extract and sorbic acid; mycosporine-like amino acid (MAA) extracts such as Porphyra Umbilicalis Extract (and) Sodium Lactate (and) Lecithin; ring cyanoacrylates such as fused-ring cyanoacrylates; and flavonoids such as Acacia Catechu Extract. Exemplary agents include ellagic acid, Porphyra Umbilicalis Extract (including Porphyra Umbilicalis Extract (and) Sodium Lactate (and) Lecithin), mycosporine-like amino acids (MAAs) and synthetic analogs thereof, Scutellaria Baicalensis Root Extract, Chrysanthemum Morifolium Leaf Extract, ferulic acid, fused-ring cyanoacrylates, curcumin, epigallocatechin gallate, Scutellaria Baicalensis Root Extract (and) Acacia Catechu Wood Extract, sorbic acid, Polygonum Cuspidatum Root Extract, Acacia Catechu Extract, dipicolinic acid, squalene, Lonicera Japonica (Honeysuckle) Flower Extract, and beta-carotene.

The agent may be an antioxidant (AOX). For example, the agent may be a peroxynitrite quencher. A peroxynitrite quencher is a chemical compound, a biological molecule, or a combination thereof that slows, decreases or inhibits oxidation of melanin by peroxynitrite. A peroxynitrite scavenging capacity (NORAC) assay may be used to identify a peroxynitrite quencher.

The agent may be a triplet energy quencher. A triplet energy quencher is a chemical compound, a biological molecule, or a combination thereof that absorbs the triplet energy generated by, for example, oxidation of melanin by peroxynitrite. A triplet energy quencher may be identified by its ability to decrease the chemiluminescence emitted by synthetic tetramethyl-1,2-dioxetane (TMD). Ethyl sorbate is a known triplet energy quencher and may be used as a positive control for identifying new triplet energy quenchers.

The agent may be a triplet energy preventer, which inhibits the generation of triplet states from, for example, melanin which is oxidized by peroxynitrite. Compounds that destroy the reaction intermediate (the dioxetane), such as vitamin E and glutathione, may be used as a triplet energy preventer.

The term “test agent” as used herein refers to an agent that is subject to screening. To identify a dCPD quencher according to the screening method of the present invention, it is preferred that the test agent does not inhibit a dioxetane from generating a triplet carbonyl, that a dioxetane is not created on the test agent, and/or that the test agent is not better than DBAS at converting triplet energy to luminescence. Vitamin E is a known dCPD blocker and may be used as a positive control for screening for new dCPD quenchers.

According to the screening method, the test agent may be identified as a dCPD quencher if it decreases the chemiluminescence signal by, for example, at least about 5%, 10%, 20%, 30%, 40%, 50%, 60%, 70%, 80%, 90%, 95%, 99% or 100%, within a predetermined period of time, for example, within about 1,000, 750, 500, 250, 200, 175, 150, 125, 100, 75, 50, 25, 10, 5 or 1 second(s), about 100-600 seconds, 125-200 seconds or 400-600 seconds, or at 150 or 500 seconds, after the test agent is added to the solvent. In one embodiment, the test agent is identified as a dCPD quencher if it decreases the chemiluminescence signal by at least about 20% within about 175 seconds after the test agent is added to the solvent.

A quenching method for slowing, decreasing or inhibiting formation of one or more dark cyclobutane pyrimidine dimers (dCPDs) in a cell or DNA molecule in a subject, who has been exposed to UV radiation, is also provided. The method comprises administering to the subject an effective amount of a dCPD quencher. The dCPD quencher may be administered to the subject within a predetermined period of time after the exposure. The predetermined period of time may be about 0.1, 0.5, 1, 2, 3, 4, 5, 6, 7 or 8 hours. The dCPD quencher may be administered to the subject in the absence of UV radiation. The dCPD quencher may decrease the formation of the one or more dCPDs in the presence of peroxynitrite and melanin.

The terms “dCPD quencher” and “dCPD inhibitor” are used herein interchangeably and refer to an agent that slows, decreases or inhibits the formation of one or more cyclobutane pyrimidine dimers (CPDs) in a cell or DNA molecule in the absence of ultraviolet (UV) radiation. The CPD is generated due to the formation of bonds between two pyrimidine bases in the DNA molecule. The DNA molecule may be single- or double-stranded. The DNA molecule may be an oligonucleotide having 2-99 nucleotides or a polynucleotide having 100 or more nucleotides. For example, the DNA molecule may be a plasmid.

The dCPD quencher may be ellagic acid, Porphyra Umbilicalis Extract (such as Porphyra Umbilicalis Extract (and) Sodium Lactate (and) Lecithin), mycosporine-like amino acids (MAAs) and synthetic analogs thereof, Scutellaria Baicalensis Root Extract, Chrysanthemum Morifolium Leaf Extract, ferulic acid, fused-ring cyanoacrylates, curcumin, epigallocatechin gallate, Scutellaria Baicalensis Root Extract (and) Acacia Catechu Wood Extract, sorbic acid, Polygonum Cuspidatum Root Extract, Acacia Catechu Extract, dipicolinic acid, squalene, Lonicera Japonica (Honeysuckle) Flower Extract, beta-carotene or a combination thereof.

The UV radiation is an electromagnetic radiation with a wavelength from 10 nm to 400 nm. The UV radiation may comprise radiation of ultraviolet A with a wavelength of 315-400 nm, ultraviolet B with a wavelength of 280-315 nm, ultraviolet C with a wavelength of 100-280 nm, or a combination thereof.

The dCPD quencher slows, decreases, or inhibits dCPD formation in a cell or DNA molecule by at least a predetermined amount, for example, about 5%, 10%, 20%, 30%, 40%, 50%, 60%, 70%, 80%, 90%, 95%, 99% or 100%, for example, by at least about 40-100%, 40-90%, 50-90%, 60-100% or 80-100%, within a predetermined period of time, for example, about 0.1, 0.5, 1, 2, 3, 4, 5, 6, 7 or 8 hours after the dCPD quencher is administered to the subject. The dCPD quencher may slow, decrease, or inhibit dCPD formation in a cell or DNA molecule in the presence of peroxynitrite and melanin. The number of CPDs may be measured by conventional techniques known in the art, for example, using an ELISA assay.

In one embodiment, the dCPD quencher decreases dCPD formation in a cell or DNA molecule in a subject within about 1, 3 or 8 hours after the subject is exposed to UV radiation by at least about 40% or 80%, within a predetermined period of time, for example, about 1 hour, after the dCPD quencher is administered to the subject.

### EXAMPLE 1. SUPEROXIDE RADICAL ABSORBANCE AND PEROXYNITRITE RADICAL ABSORBANCE CAPACITY TESTS

Superoxide radical absorbance capacity tests were conducted to measure the capacity of an antioxidant material to dismutate superoxide anion. The Varioskan Flush was employed to measure the superoxide dismutase-catalyzed dismutation of the superoxide anion in the presence of the absorbance probe, water-soluble tetrazolium salt (WST-1). The superoxide radical antioxidant (SORAC) assay is based on an inhibition assay and is suitable for evaluating superoxide dismutase or superoxide dismutase-like activities in samples.

Peroxynitrite radical absorbance capacity tests were conducted to measure the capacity of an antioxidant material to quench peroxynitrite. Dihydrorhodamine 123 was used as an indicator of peroxynitrite in tubo for peroxynitrite radical absorbance capacity tests with Varioskan Flush.

The antioxidants tested here are the top performers of anti-peroxynitrite based on screening of more than 200 antioxidants. The test results are illustrated in Table 1.

TABLE 1

| | Anti- | Anti- |
|---|---|---|
| Antioxidant Material | superoxide | peroxynitrite |
| Ellagic acid | 2 | 4 |
| Epigallocatechin Gallate | 4 | 4 |
| Scutellaria Baicalensis Root Extract | 2 | 4 |
| (and) Acacia Catechu Wood Extract | | |
| Chrysanthemum Morifolium Leaf Extract | 4 | 4 |
| Scutellaria Baicalensis Root Extract | 2 | 4 |
| Ferulic Acid | 1 | 4 |
| Resveratrol | 0 | 4 |
| Polygonum Cuspidatum Root Extract | 0 | 3 |
| Acacia Catechu Wood Extract (And) | 3 | 4 |
| Scutellaria Baicalensis Root Extract | | |
| Lonicera Japonica (Honeysuckle) | 2 | 4 |
| Flower Extract | | |
| Acacia Catechu Extract | 3 | 4 |
| Ampelopsis Extract | 3 | 4 |

0 No activity
1 Low activity
2 Medium activity
3 Strong activity
4 Super strong activity

### EXAMPLE 2. MELANIN OXIDATION ASSAY

An easy and fast assay was developed to measure quenching of chemiluminescence from the triplet carbonyl of a cleaved dioxetane that was created when peroxynitrite (ONOO—) reacted with melanin. As such, it measured both triplet quenching and any scavenging activity that prevented the chemiexcitation reaction from starting. DMSO was used as a solvent for the entire reaction, DBAS to enhance the luminescence and a liquid scintillation counter set to single-photon mode to detect the luminescence signal.

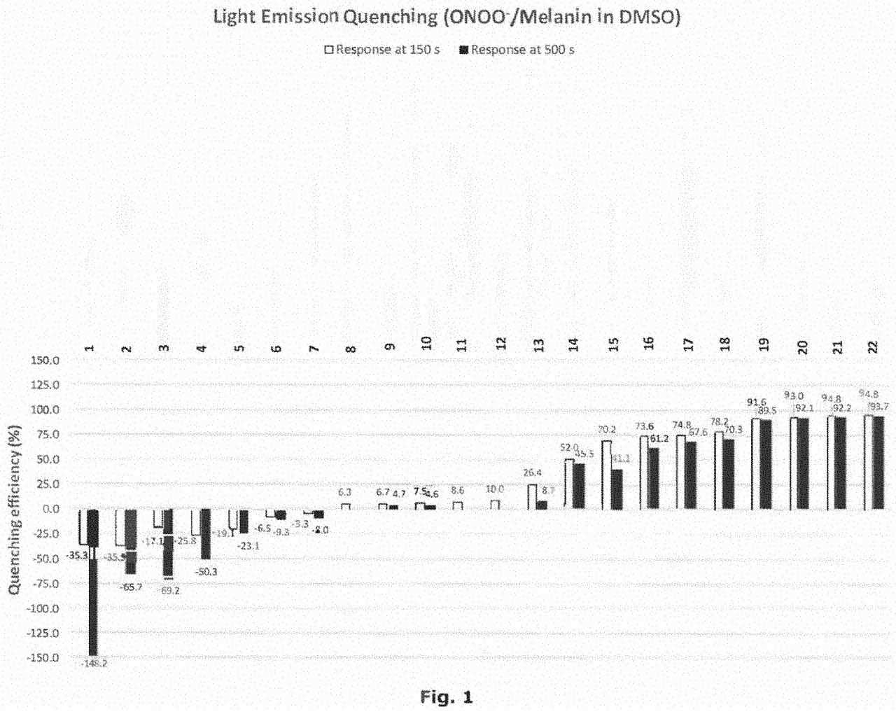

In this assay, peroxynitrite, melanin, and DBAS were incubated in DMSO in the absence or presence of test agents, and light emission quenching (%) was measured at 150 seconds and 500 seconds (FIG. 1). The test agents included ampelopsis extract, trimethoxybenzylidene pentanedione, resveratrol, acacia extract, vit E (vitamin E), retinol, Polyester-25, ethylhexyl methoxycrylene, Polygonum Cuspidatum Root Extract, squalene, Lonicera Japonica (Honeysuckle) Flower Extract, Acacia Catechu Wood Extract (and) Scutellaria Baicalensis Root Extract, beta-carotene, Scutellaria Baicalensis Root Extract (and) Acacia Catechu Wood Extract, fused-ring cyanoacrylates, epigallocatechin gallate, ferulic acid, Chrysanthemum Morifolium Leaf Extract, sorbic acid, Scutellaria Baicalensis Root Extract, dipicolinic acid, curcumin and ellagic acid. Hits that showed a quenching effect included Scutellaria Baicalensis Root Extract (and) Acacia Catechu Wood Extract, fused-ring cyanoacrylates, epigallocatechin gallate, ferulic acid, Chrysanthemum Morifolium Leaf Extract, sorbic acid, Scutellaria Baicalensis Root Extract, dipicolinic acid, curcumin, ellagic acid, Polygonum Cuspidatum Root Extract, squalene, Lonicera Japonica (Honeysuckle) Flower Extract, and beta-carotene.

It is noted that a hit can also come from a compound that chemically inactivates the dioxetane, that chemiluminescence could increase above a positive control if ONOO— creates a dioxetane on the compound or the compound is better than DBAS at converting triplet energy to luminescence.

### EXAMPLE 3. DARK CPD QUENCHER “HITS” VALIDATION

A dark CPD quenching assay was carried out to determine whether a hit identified in Example 1 could quench dark CPD. In this assay, a polynucleotide having the sequence of (dT)₁₀₀ was incubated alone (Oligo only) or with melatonin (Oligo+melatonin), peroxynitrite (ONOO), melatonin and peroxynitrite (Oligo+melatonin+ONOO), or melatonin, peroxynitrite and a “hit” from Example 1. Because the CPD quenching assay was done in an aqueous buffer, in which melanin is not soluble, the related model compound melatonin was used instead. The hits included ellagic acid, a polyphenol such as epigallocatechin gallate, flavones such as Scutellaria Baicalensis Root Extract (and) Acacia Catechu Wood Extract, Chrysanthemum Morifolium Leaf Extract and Scutellaria Baicalensis Root Extract, cinnamate-related compounds such as curcumin, ferulic acid and sorbic acid, mycosporine- like amino acid (MAA) extracts such as Porphyra Umbilicalis Extract (and) Sodium Lactate (and) Lecithin, and fused-ring cyanoacrylates. Exemplary test agents include ellagic acid, Porphyra Umbilicalis Extract (and) Sodium Lactate (and) Lecithin (which includes mycosporine-like amino acids, MAAs), Scutellaria Baicalensis Root Extract, Chrysanthemum Morifolium Leaf Extract, ferulic acid, fused-ring cyanoacrylates, curcumin, epigallocatechin gallate, Scutellaria Baicalensis Root Extract (and) Acacia Catechu Wood Extract, and sorbic acid.

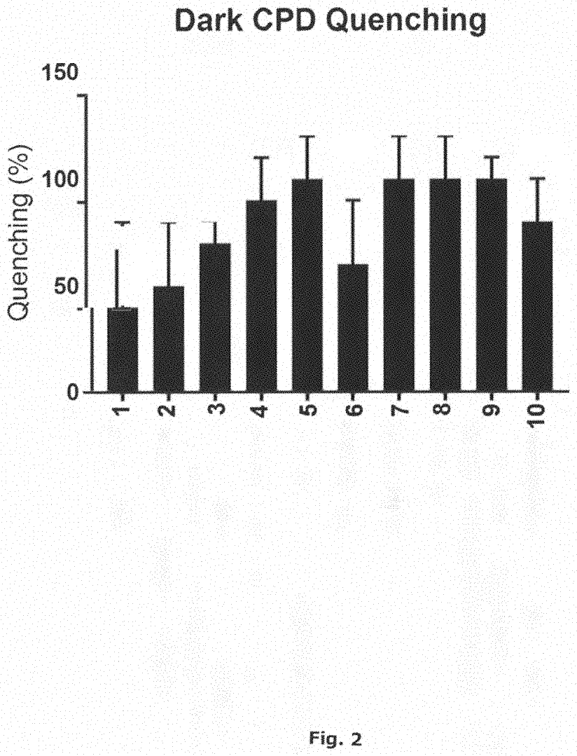

The dark CPD quenching results confirmed that “hits” identified in the melanin oxidation assay as described in Example 2 also quenched dark CPD by 40-100% (FIG. 2).

Not all peroxynitrite quenchers were validated as dCPD quenchers. Only some (not all) of the tested highest performing NORAC peroxynitrite quenchers were effective in the CPD quencher assay.

Although the invention is illustrated and described herein with reference to specific embodiments, the invention is not intended to be limited to the details shown. Rather, various modifications may be made in the details within the scope and range of equivalents of the claims without departing from the invention.

What is claimed:

1. A method for decreasing formation of one or more chemiexcitation-induced dark cyclobutane pyrimidine dimers (dCPDs) in a cell or DNA molecule in a subject who has been exposed to UV radiation, comprising administering to the subject an effective amount of a dCPD quencher within 8 hours after the exposure, wherein the dCPD quencher is selected from the group consisting of ellagic acid, Porphyra Umbilicalis Extract, mycosporine-like amino acids (MAAs) and synthetic analogs thereof, Scutellaria Baicalensis Root Extract, Chrysanthemum Morifolium Leaf Extract, ferulic acid, fused-ring cyanoacrylates, curcumin, epigallocatechin gallate, Scutellaria Baicalensis Root Extract (and) Acacia Catechu Wood Extract, sorbic acid, Polygonum Cuspidatum Root Extract, Acacia Catechu Extract, dipicolinic acid, squalene, Lonicera Japonica (Honeysuckle) Flower Extract, beta-carotene and combinations thereof.

2. The method of claim 1, wherein the dCPD quencher is administered to the subject within 3 hours after the exposure.

3. The method of claim 1, wherein the dCPD quencher is administered to the subject within 1 hour after the exposure.

4. The method of claim 1, wherein the dCPD quencher is administered to the subject in the absence of UV radiation.

5. The method of claim 1, further comprising decreasing the formation of the one or more dCPDs by at least 40%.

6. The method of claim 1, further comprising decreasing the formation of the one or more dCPDs by at least 80%.

7. The method of claim 1, further comprising decreasing the formation of the one or more dCPDs within 1 hour after the dCPD quencher is administered to the subject.

8. The method of claim 1, wherein the dCPD quencher is ellagic acid.

9. The method of claim 1, wherein the dCPD quencher is Porphyra Umbilicalis Extract (and) Sodium Lactate (and) Lecitchin.

10. The method of claim 1, wherein the dCPD quencher is one or more mycosporine-like amino acids (MAAs) or synthetic analogs thereof.

11. The method of claim 1, wherein the dCPD quencher is Scutellaria Baicalensis Root Extract.

12. The method of claim 1, wherein the dCPD quencher is Chrysanthemum Morifolium Leaf Extract.

13. The method of claim 1, wherein the dCPD quencher is ferulic acid.

14. The method of claim 1, wherein the dCPD quencher is fused-ring cyanoacrylates.

15. The method of claim 1, wherein the dCPD quencher is curcumin.

16. The method of claim 1, wherein the dCPD quencher is epigallocatechin gallate.

17. The method of claim 1, wherein the dCPD quencher is Scutellaria Baicalensis Root Extract (and) Acacia Catechu Wood Extract.

18. The method of claim 1, wherein the dCPD quencher is sorbic acid.

19. The method of claim 1, wherein the dCPD quencher is Polygonum Cuspidatum Root Extract.

20. The method of claim 1, wherein the dCPD quencher decreases the formation of the one or more dCPDs in the presence of peroxynitrite and melanin.
